(19) United States
(12) Patent Application Publication
(10) Pub. No.: US 2026/0083352 A1
(43) Pub. Date: Mar. 26, 2026

(54) MEASURING BODY PARTS USING TOUCHSCREEN DISPLAYS

(71) Applicant: My First Steps LLC, Wilmington, DE (US)

(72) Inventor: Daniel Delgadillo, III, Hermosa Beach, CA (US)

(21) Appl. No.: 18/895,043

(22) Filed: Sep. 24, 2024

(51) Int. Cl. A61B 5/107; A61B 5/00; A61B 5/053

(52) U.S. Cl. CPC A61B 5/1075; A61B 5/053; A61B 5/1074; A61B 5/1079; A61B 5/7267; A61B 5/748

(57) ABSTRACT

An example apparatus for measuring human body parts includes a touchscreen display to detect touch capacitance information associated with a body part. The apparatus also includes a processor to determine a size of the body part based on the detected touch capacitance information.

### BACKGROUND

Personal electronic devices (PEDs) often include touchscreen displays that are capable of detecting touch inputs from users. Touchscreen displays generally include an input device known as a touchscreen panel, and an output device that is a visual display. The touchscreen panel is typically layered on top of the visual display, which may be a liquid crystal display (LCD), active-matrix organic light-emitting diode (AMOLED), or an organic light-emitting diode (OLED) display.

The touchscreen panels of most modern PEDs are capacitive touchscreen panels. A capacitive touchscreen panel typically includes an insulator, such as glass, coated with a transparent conductor, such as indium tin oxide (ITO) or silver. Touching the surface of the screen with an electrical conductor such as human skin results in a distortion of the screen's electrostatic field, which is measurable as a change in capacitance.

### BRIEF DESCRIPTION OF THE DRAWINGS

FIG. 1 is a block diagram illustrating an example system for measuring body parts using touchscreen displays;

FIG. 2A is a diagram illustrating an example personal electronic device measuring the size of a foot using a touchscreen display, according to embodiments herein;

FIG. 2B is a diagram illustrating an example personal electronic device measuring the size of a foot using a touchscreen display with a digital ruler, according to embodiments herein;

FIG. 3A is a process flow diagram illustrating a method for measuring body parts using touchscreen displays;

FIG. 3B is a process flow diagram illustrating a method for measuring body parts using touchscreen displays;

FIG. 3C is a process flow diagram illustrating a method for measuring body parts using digital prints;

FIG. 3D is a process flow diagram illustrating a method for measuring body parts using a temperature safety device;

FIG. 3E is a process flow diagram illustrating a method for generating recommendations or verifying body part sizes using touchscreen displays;

FIG. 4 is a process flow diagram illustrating a method for training a neural network to predict body part sizes;

FIG. 5 is block diagram illustrating an example computing device that can measure body parts using touchscreen displays;

FIG. 6 is a block diagram showing computer readable media that store code for measuring body parts using touchscreen displays; and

FIG. 7 is a process flow diagram illustrating an example process for generating a digital print of a foot.

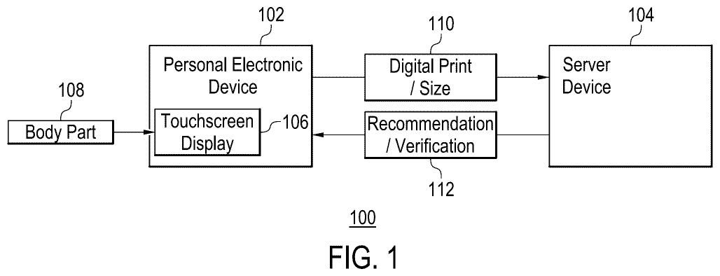

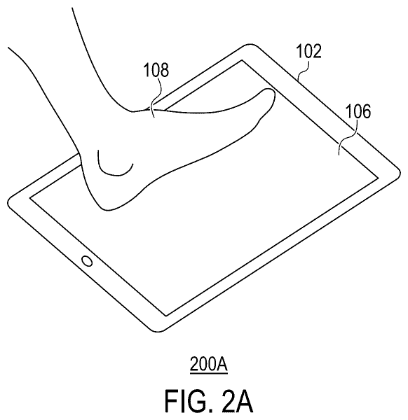

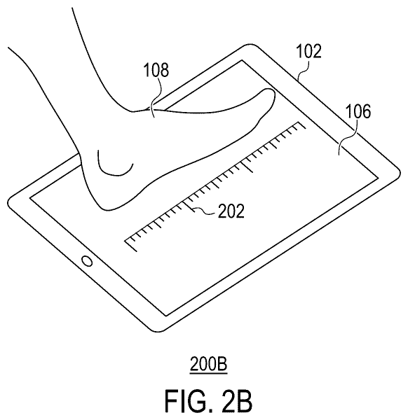

The same numbers are used throughout the disclosure and the figures to reference like components and features. Numbers in the 100 series refer to features originally found in FIG. 1; numbers in the 200 series refer to features originally found in FIG. 2; and so on.

### DESCRIPTION OF THE EMBODIMENTS

Human body parts may often need to be measured for various reasons. However, such measurements may often be difficult for many people. Measurements of the body parts of their infants or toddlers may be particularly difficult. For example, many parents currently struggle with measuring the feet of their younger children. Specifically, parents may have difficulty accurately measuring the length and/or width of the foot of an infant or toddler. And without an accurate measurement, parents may also have difficulties determining the proper size of shoes for their children. Similar measurement issues may also arise when attempting to measure the size of hands for gloves, or other body parts for corresponding form-fitting products.

Some methods to measure feet of infants and toddlers that currently exist include string-based methods, which generally involve the placement of a string along the plantar surface of the foot, followed by measuring the length of the string using any suitable measuring device. However, this method is difficult to use when evaluating the width of the foot. Moreover, various physical conditions can similarly make measurement of the length of hands or feet difficult.

Another method of determining shoe size for an infant or toddler includes purchasing multiple sizes (including combinations of different lengths and widths) of shoes, and returning sizes that are inappropriate. However, this method places a large and unnecessary burden on shoe companies, shoe stores, and shipping companies.

With the growing ownership of touchscreen personal electronic devices worldwide, access to touchscreen displays is now common. However, measuring body parts via touchscreen interaction also has technical issues. For example, the portion of the body part physically touching the touchscreen may not be the same size as the body part itself. Moreover, not all body parts are physically the same. For example, some body parts may have special features that do not conform to a single body part model. In addition, in some instances, the touchscreens may be too hot to press particularly sensitive body parts to safely take a measurement.

The present disclosure utilizes such touchscreen displays to solve the aforementioned problems. In some embodiments, a digital print is generated from touch capacitance information received from the touchscreen panel and used to automatically determine an accurate size of an associated body part. As used herein, touch capacitance information refers to data received from a touchscreen panel. For example, touch capacitance information includes raw events, such as coordinates of detected touchpoints, the precision of the coordinates, and associated pressure, size, the number of pointers in an event, and the number of historical points in an event. As used herein, a digital print refers to a two-dimensional outline of a body part associated with touch capacitance information. The resulting size may be used to accurately fit infant and toddler feet with appropriately sized shoes. Additionally, aspects of the present disclosure may aid in identifying children with foot size discrepancies that require custom shoes. For example, some children may have a left foot with a different size than their right foot, which may include length and/or width differences. Moreover, such measurements could also be used to develop customized shoes for children with various physical conditions. For example, such physical conditions may include clubfoot, also known as talipes equinovarus, a birth defect in which the foot and ankle are twisted out of shape or position. Another physical condition includes flat feet (pes planus), in which the normal arch in the middle of the feet appears flattened. In contrast, some feet may have high arches, also known as cavus foot, in which the arch of the foot is raised more than normal. For infants and toddlers with such conditions, improperly sized shoes may potentially cause harm. For example, a size difference may cause foot growth and or pain that toddlers may be unable to express. With proper measurements, customized shoes can be designed for any such feet. In some embodiments, the aspects of the present disclosure can be used to provide proper fitting gloves for hands, which may be very useful for those purchasing gloves for sports such as golf, football, tennis, etc. In other embodiments, aspects of the present disclosure may also be used to verify the size of a body part against a purported size. This may be helpful in situations where two individuals are remotely interacting and the size of the body party cannot be accurately verified in person. For example, the size of a body part may appear very different depending on the distance of the body part from a camera.

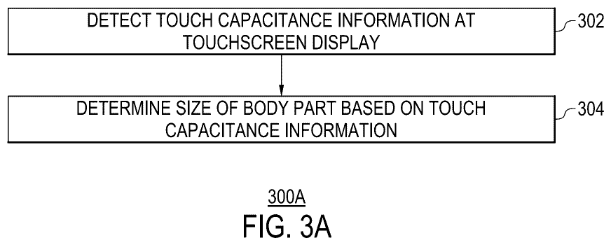

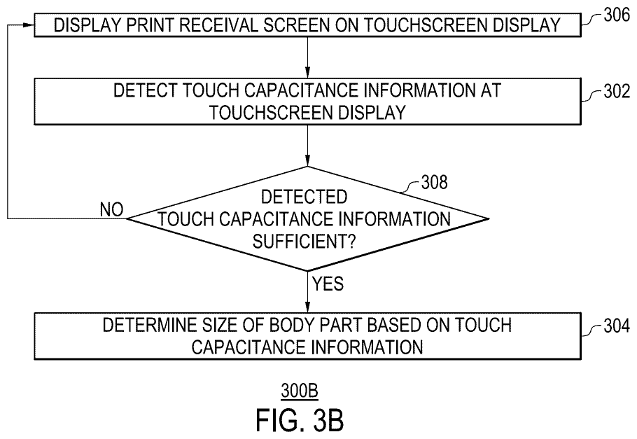

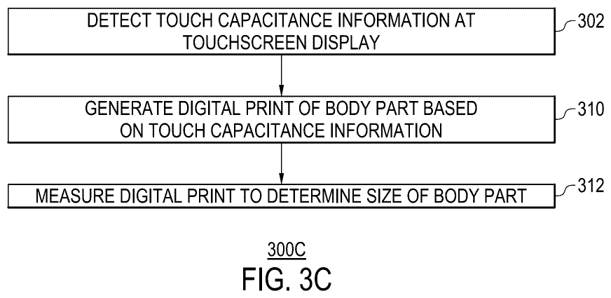

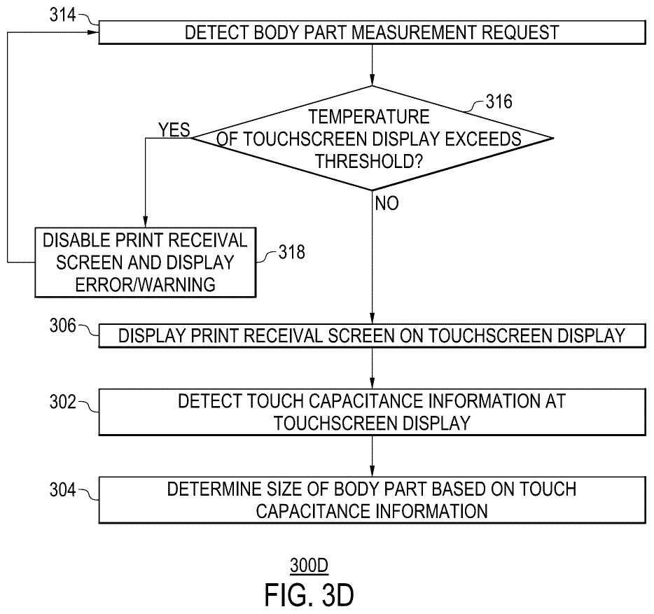

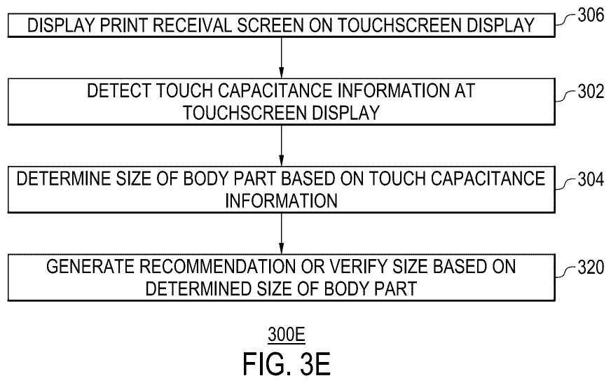

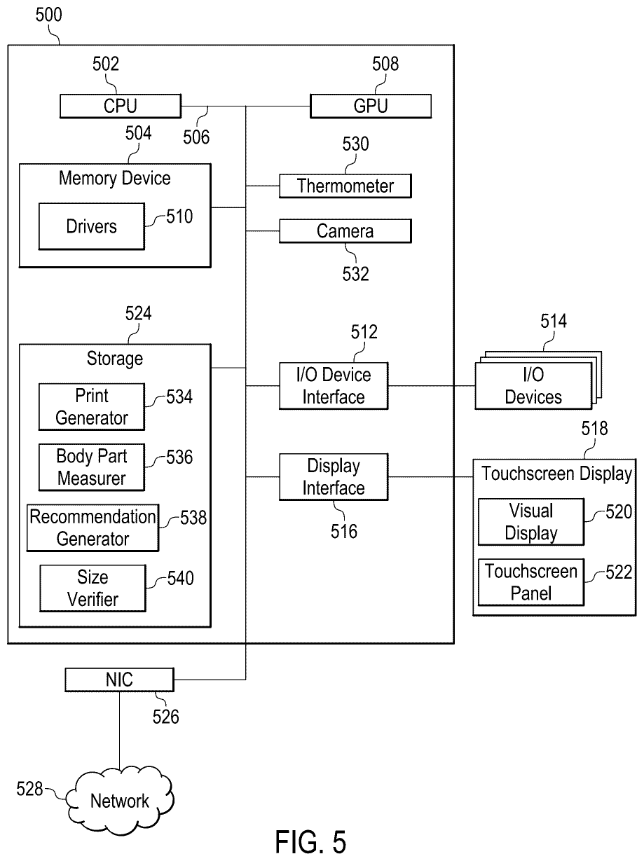

FIG. 1 is a block diagram illustrating an example system for measuring body parts using touchscreen displays. The example system 100 can be implemented in the computing device 500 of FIG. 5 using any combination of the methods 300A-300E of FIGS. 3A-3E.

The example system 100 includes a personal electronic device (PED) 102 communicatively coupled to a service device 104. In various embodiments, the PED 102 includes a touchscreen display 106. For example, the PED 102 may be a phone or a tablet device that includes a touchscreen display 106.

In various embodiments, the PED 102 receives a body part 108 at the touchscreen display 106. For example, the body part 108 may be a foot, a hand, or any other extremity, such a wrists, fingers, or penises, among other anatomical parts. As described in greater detail below, the PED 102 can generate a digital print based on touch capacitance information received from the touchscreen display 106. In some embodiments, the PED 102 can also measure a size of the body part based on any suitable metric. In various examples, the size of the body part can be measured using the digital print, the touch capacitance information, an image of the body part, or any combination thereof.

In various embodiments, the server device 104 receives the digital print or size 110 from the PED 102. In some embodiments, the server device 104 receives a digital print and determines a size of the body part based on the digital print. In these embodiments, the server device 104 can also generate a recommendation based on the determined size, such as a product or service. In one embodiment, the product is a shoe, with appropriate width and length for a foot corresponding to the digital print. In some embodiments, the server device 104 generates a verification. For example, the server device 104 can verify that a received purported size of the body part matches the received digital print generated by the PED 102.

In some embodiments, the server device 104 receives a size of the body part. For example, the size of the body part may be a two-dimensional measurement in inches or centimeters. In various examples, the size may include a length of the part, a width of the part. As one example, the width of a body part may be determined as the measured maximum width along a vertical axis corresponding to the length of the body part. The length may be measured based on a distance between two detected features. For example, the length of a foot may be the distance between the tip of the big toe and base of the heel, as measured along a line that is perpendicular to the measured width of the foot. The server device 104 can then provide a recommendation based on the received size. For example, the recommendation may be a particular shoe that properly fits a measured foot's length, width, or both. In some examples, shoes with different sizes may be recommended. For example, the right foot of an individual may be differently sized than the left foot of an individual. In some examples, shoes specifically developed for special needs may be recommended. For example, shoes with high arch support may be recommended in response to detecting that the digital print is associated with high arches. In various examples, the server device 104 can generate and send a verification to the PED 102. For example, the verification may confirm that a purported size of a body part matches the size indicated by the size determined by the PED 102.

The diagram of FIG. 1 is not intended to indicate that the example system 100 is to include all of the components shown in FIG. 1. Rather, the example system 100 can be implemented using fewer or additional components not illustrated in FIG. 1 (e.g., additional PEDs, server devices, digital prints, sizes, recommendations, verifications, displays, body parts, types of body parts, etc.).

FIG. 2A is a diagram illustrating an example personal electronic device measuring the size of a foot using a touchscreen display. The example personal electronic device 200A can be implemented in the computing device 500 in FIG. 5 using the methods 300A-300E of FIGS. 3A-3E.

The example personal electronic device 102 includes similarly referenced elements from FIG. 1. In particular, the PED 102 includes a display 106, upon which a body part 108 is placed in capacitive communication therewith. In the example of FIG. 2A, the body part is a foot 108 of a toddler and the PED 102 a cellphone with a capacitive touchscreen display 106. In various embodiments, the display 106 may include a prompt to place the body part 108 within a specific region of the display 106. In some examples, the body part 108.

The diagram of FIG. 2A is not intended to indicate that the example personal electronic device 200A is to include all of the components shown in FIG. 2A. Rather, the example personal electronic device 200A can be implemented using fewer or additional components not illustrated in FIG. 2A (e.g., additional devices, displays, body parts, types of body parts, etc.). Moreover, in various embodiments, the position of the foot on the device may be in different locations of the PED 102 or rotated in any direction with respect to the PED 102.

FIG. 2B is a diagram illustrating an example personal electronic device 200B measuring the size of a foot using a touchscreen display with a digital ruler 202. In various embodiments, the personal electronic device 200B can be implemented in the computing device 500 in FIG. 5 using the methods 300A-300E of FIGS. 3A-3E.

The example personal electronic device 200B includes similarly referenced elements described in FIG. 2A. For example, the PED 102 includes a display 106, upon which a body part 108 is placed in capacitive communication therewith. In addition, the example personal electronic device 200B includes a ruler 202. In various embodiments, a ruler 202 provides users an estimated visual size of the body part 108. In some embodiments, the ruler 202 can be used to guide the body part 108 to a particular portion of the PED 102. For example, in instances where the display 106 may be occluded by one or more features of the PED 102, the ruler 202 can be used to guide a user to place a body part 108 towards a less occluded portion of the screen to ensure a more accurate and full digital print of the body part.

The diagram of FIG. 2B is not intended to indicate that the example personal electronic device 200B is to include all of the components shown in FIG. 2B. Rather, the example personal electronic device 200B can be implemented using fewer or additional components not illustrated in FIG. 2B (e.g., additional devices, displays, types of visual guides, body parts, types of body parts, etc.). Moreover, in various embodiments, the position of the foot on the device may be in different locations of the PED 102 or rotated in any direction with respect to the PED 102.

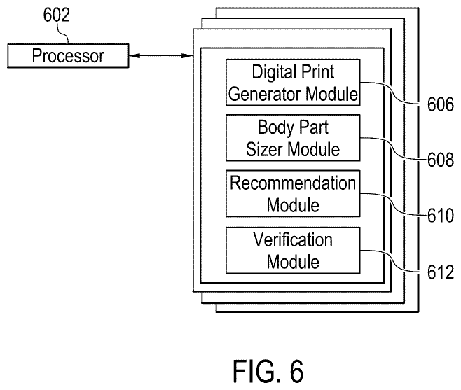

FIG. 3A is a process flow diagram illustrating a method 300A for measuring body parts using touchscreen displays. The example method 300A can be implemented in the system 100 of FIG. 1, the computing device 500 of FIG. 5, or the computer readable media 600 of FIG. 6.

At block 302, touch capacitance information is detected at a touchscreen display. In various embodiments, the touchscreen display is a capacitive touchscreen. In some embodiments, a smoothing or filtering of the touch capacitance information may be executed to handle minor inaccuracies in touch detection.

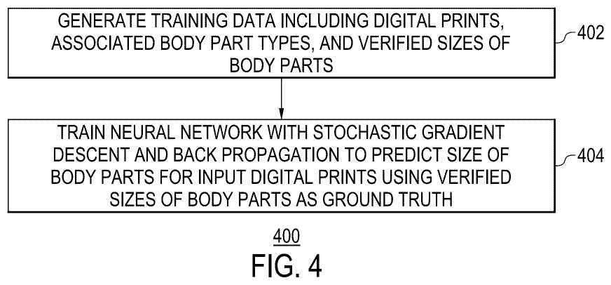

At block 304, a size of a body part is determined based on touch capacitance information. For example, the touch capacitance information can include raw data received from a touchscreen panel. In some embodiments, the size of the body part is determined using a neural network. As used herein, a neural network refers to an artificial neural network, which is a type of machine learning model used to perform a wide variety of complex tasks, including image recognition, speech recognition, pattern recognition, and detection of anomalies. A neural network is a biologically inspired algorithm that learns from training data. A neural network can be realized through software, hardware, or a combination of software and hardware. The structure of an exemplary neural network has a series of layers, each comprising one or more neurons arranged in one or more neuron arrays. In an exemplary embodiment, a neuron may include a register, a microprocessor, and at least one input. Each neuron produces an output, or activation, based on an activation function that uses the outputs of the previous layer and a set of weights as inputs. Each neuron in a neuron array may be connected to another neuron via a synaptic circuit. A synaptic circuit may include a memory for storing a synaptic weight. An exemplary neural network may be a Deep Neural Network having an input layer, an output layer, and any number of fully connected hidden layers. Neural networks may be particularly useful in the sizing of body parts because they can effectively extract features in linear and nonlinear relationships. In some embodiments, a neural network may be implemented by an application-specific integrated circuit (ASIC). For example, ASICs may be specially customized for a specific artificial intelligence application and provide improved computing capabilities and reduced electricity consumption compared to traditional CPUs. In various embodiments, the neural network may be trained to receive touch capacitance information and output a predicted size of the body part. In some embodiments, the neural network is trained to receive a digital print and output a corresponding body part size. For example, the neural network may have been trained using stochastic gradient descent, backward propagation, as described in FIG. 4.

This process flow diagram is not intended to indicate that the blocks of the example method 300 are to be executed in any particular order, or that all of the blocks are to be included in every case. Further, any number of additional blocks not shown may be included within the example method 300, depending on the details of the specific implementation. For example, in addition to the touch capacitance information, in some embodiments, images of the body part captured using the same electronic device or another electronic device may also be used to determine the size of the body part.

FIG. 3B is a process flow diagram illustrating another method 300B for measuring body parts using touchscreen displays. The example method 300B can be implemented in the system 100 of FIG. 1, the computing device 500 of FIG. 5, or the computer readable media 600 of FIG. 6.

FIG. 3B includes similarly referenced elements from FIG. 3A. For example, at block 302, touch capacitance information is detected at a touch display. At block 304, the size of a body part is determined based on touch capacitance information.

In addition, at block 306, a print receival screen is displayed on a touchscreen display. For example, the print receival screen may be displayed in response to detecting a request to measure a body part. The print receival screen may include information such as instructions as to how to properly present a body part to the touchscreen display for measurement. In some embodiments, the print receival screen also includes guide markers that may be used to guide a user to place a body part onto a specified portion of the screen. In one embodiment, the guide marker may be in the form of a ruler positioned at one side or the middle of the touchscreen display. In some embodiments, different sizes of a detected body part can be outlined on the screen based on the determined size of the digital print. For example, the outlined size of the current digital print may be displayed, along with one size up and down, with a toggle to change the size from regular width to wide width.

At decision diamond 308, a determination is made as to whether the detected touch capacitance information is sufficient. For example, the determination may include a check to verify that a threshold number of features are present in the touch capacitance information. In various embodiments, if the determination results in detecting that the touch capacitance information is sufficient, then the method 300B may continue at block 304. Otherwise, if the determination is made that the touch capacitance information is not sufficient, then the method 300B may continue at block 306. For example, the print receival screen may again be displayed in order to receive a follow-up presentation of the body part.

This process flow diagram is not intended to indicate that the blocks of the example method 300B are to be executed in any particular order, or that all of the blocks are to be included in every case. Further, any number of additional blocks not shown may be included within the example method 300B, depending on the details of the specific implementation.

FIG. 3C is a process flow diagram illustrating a method 300C for measuring body parts using digital prints. The example method 300C can be implemented in the system 100 of FIG. 1, the computing device 500 of FIG. 5, or the computer readable media 600 of FIG. 6.

FIG. 3C includes similarly referenced elements from FIG. 3A. For example, at block 302, touch capacitance information is detected at a touch display.

At block 310, a digital print of a body part is generated based on the touch capacitance information. For example, the digital print may be a two-dimensional outline of the body part. In various examples, the digital print does not include personally identifying information, such as fingerprints, thumbprints, etc.

At block 312, the digital print is measured to determine a size of the body part. For example, the digital print may be matched with a particular model for the body part and then measured and converted into a measurement of the body part. In some embodiments, the digital print can alternatively be input into a trained neural network to receive a size of the body part from the trained neural network.

This process flow diagram is not intended to indicate that the blocks of the example method 300C are to be executed in any particular order, or that all of the blocks are to be included in every case. Further, any number of additional blocks not shown may be included within the example method 300C, depending on the details of the specific implementation. For example, in some embodiments, the digital print is displayed on the touchscreen display. Displaying the digital print on the touchscreen display may provide feedback with respect to digital print capture and thus improve usability of the device.

FIG. 3D is a process flow diagram illustrating a method 300D for measuring body parts using a temperature safety device. The example method 300D can be implemented in the system 100 of FIG. 1, the computing device 500 of FIG. 5, or the computer readable media 600 of FIG. 6.

FIG. 3D includes similarly referenced elements from FIGS. 3A and 3B. For example, at block 302, touch capacitance information is detected at a touch display. At block 304, the size of a body part is determined based on touch capacitance information. At block 306, a print receival screen is displayed on a touchscreen display.

At block 314, a body part measurement request is detected. For example, the body part measurement request may be received from an application on a personal electronic device associated with the touchscreen display.

At decision diamond 316, a determination is made as to whether a temperature of a touchscreen display exceeds a threshold. For example, the temperature of the touchscreen display may be too hot to safely use as a measurement device for one or more body parts. In various examples, the threshold may be based on the specific body part to be measured, or the age of the person whose body part is to be measured. For example, a lower threshold may be used for infants or toddlers to prevent heat damage to more sensitive skin.

At block 318, a print receival screen is disabled and an error/warning is displayed. For example, the error/warning may include information such as the heat status of the device. In some examples, the error/warning may include information such as instructions to wait a predetermined amount of time before sending another measurement request.

This process flow diagram is not intended to indicate that the blocks of the example method 300D are to be executed in any particular order, or that all of the blocks are to be included in every case. Further, any number of additional blocks not shown may be included within the example method 300D, depending on the details of the specific implementation.

FIG. 3E is a process flow diagram illustrating a method 300E for generating recommendations or verifying body part sizes using touchscreen displays. The example method 300E can be implemented in the system 100 of FIG. 1, the computing device 500 of FIG. 5, or the computer readable media 600 of FIG. 6.

FIG. 3E includes similarly referenced elements from FIGS. 3A and 3B. For example, at block 302, touch capacitance information is detected at a touch display. At block 304, the size of a body part is determined based on touch capacitance information. At block 306, a print receival screen is displayed on a touchscreen display.

In addition, at block 320, a recommendation is generated or a size is verified based on the determined size of the body part. For example, the recommendation may be a specific shoe size for a particular foot. In some embodiments, the shoe size may include a length, a width, or both. In various embodiments, a purported size of a body part is verified based on the determined size of the body part. For example, a target size may be received and the determined size of the body part compared with the target size to determine whether the body part meets or exceeds the target size. Alternatively, in some embodiments, the target size may be a maximum size, and the verification may be performed to determine whether the determined size does not exceed the target size.

This process flow diagram is not intended to indicate that the blocks of the example method 300E are to be executed in any particular order, or that all of the blocks are to be included in every case. Further, any number of additional blocks not shown may be included within the example method 300E, depending on the details of the specific implementation. For example, any combination of blocks from methods 300A-300D may also be included.

FIG. 4 is a process flow diagram illustrating a method for training a neural network to predict body part sizes. The example method is generally referred to by the reference number 400 and can be implemented in the system 100 of FIG. 1, the computing device 500 of FIG. 5, or the computer readable media 600 of FIG. 6.

At block 402, training data including digital prints, associated body part types, and verified size of the body parts is generated. For example, the digital prints may be generated using any combination of various computer devices having a capacitive touchscreen display. In some embodiments, the digital prints may be associated with one particular computer device, such as a specific model of a personal electronic device. In some examples, touch capacitance information can be used instead of generated digital prints. For example, the touch capacitance information may include raw data from the touchscreen panel associated with a digital print generated for a particular body part on a particular device.

At block 404, a neural network is trained using stochastic gradient descent and back propagation to predict sizes of various body parts for input digital prints using the verified sizes of the body parts as ground truth. Gradient descent is an optimization algorithm used to minimize differentiable real-valued multivariate functions. Gradient descent begins by initializing the values of parameters and then applying a gradient descent calculation, which uses mathematical calculations to iteratively adjust the values to minimize a loss function to optimize the neural network. Backpropagation is the mathematical process of calculating the derivatives and gradient descent is the process of adjusting model parameters using the calculated derivatives to minimize the loss function. Backpropagation is a mathematical calculation for supervised learning of neural networks using gradient descent. Given a neural network and an error function, backpropagation can be used to calculate the gradient of the error function with respect to the weights of the neural network. The generated neural network may then be used to determine a size of a body part associated with a received digital print that is not part of the training data, as described in FIG. 3C. In various embodiments, a neural network may be trained for each specific model of device. In some embodiments, a single neural network may be trained for use on multiple devices. For example, the neural network may be trained to measure body parts using screen resolution and pixel density of the touchscreen panel as additional inputs.

This process flow diagram is not intended to indicate that the blocks of the example method 400 are to be executed in any particular order, or that all of the blocks are to be included in every case. Further, any number of additional blocks not shown may be included within the example method 400, depending on the details of the specific implementation. For example, in some embodiments, the neural network may be trained with additional inputs, such as images of body parts corresponding to the digital prints. In these embodiments, the neural network can be trained to detect special conditions such as high arches in feet using the additional input image captured by a camera of the personal electronic device.

Referring now to FIG. 5, a block diagram is shown illustrating an example computing device that can measure body parts using touchscreen displays. In various examples, the computing device 500 may be any suitable device containing a capacitive touchscreen display. For example, the computing device 500 may be a mobile device, such as a tablet computer, or phone, among other devices. The computing device 500 may include a central processing unit (CPU) 502 that is configured to execute stored instructions, as well as a memory device 504 that stores instructions that are executable by the CPU 502. The CPU 502 may be coupled to the memory device 504 by a bus 506. Additionally, the CPU 502 can be a single core processor, a multi-core processor, a computing cluster, or any number of other configurations. Furthermore, the computing device 500 may include more than one CPU 502. In some examples, the CPU 502 may be a system-on-chip (SoC) with a multi-core processor architecture. In some examples, the CPU 502 can be a specialized digital signal processor (DSP) used for image processing. The memory device 504 can include random access memory (RAM), read only memory (ROM), flash memory, or any other suitable memory systems. For example, the memory device 504 may include dynamic random access memory (DRAM).

The memory device 504 can include random access memory (RAM), read only memory (ROM), flash memory, or any other suitable memory systems. For example, the memory device 504 may include dynamic random access memory (DRAM).

The computing device 500 may also include a graphics processing unit (GPU) 508. As shown, the CPU 502 may be coupled through the bus 506 to the GPU 508. The GPU 508 may be configured to perform any number of graphics operations within the computing device 500. For example, the GPU 508 may be configured to render or manipulate graphics images, graphics frames, videos, or the like, to be displayed to a user of the computing device 500.

The memory device 504 can include random access memory (RAM), read only memory (ROM), flash memory, or any other suitable memory systems. For example, the memory device 504 may include dynamic random access memory (DRAM). The memory device 504 may include device drivers 510 that are configured to execute the instructions for training multiple convolutional neural networks to perform sequence independent processing. The device drivers 510 may be software, an application program, application code, or the like.

The CPU 502 may also be connected through the bus 506 to an input/output (I/O) device interface 512 configured to connect the computing device 500 to one or more I/O devices 514. The I/O devices 514 may include, for example, a keyboard and a pointing device, wherein the pointing device may include a touchpad or a touchscreen, among others. The I/O devices 514 may be built-in components of the computing device 500, or may be devices that are externally connected to the computing device 500. In some examples, the memory 504 may be communicatively coupled to I/O devices 514 through direct memory access (DMA).

The CPU 502 may also be linked through the bus 506 to a display interface 516 configured to connect the computing device 500 to a touchscreen display device 518. The touchscreen display device 518 may include a visual display screen that is a built-in component of the computing device 500. In some embodiments, the touchscreen display device 518 may also include a computer monitor, television, among others, that is internal to or externally connected to the computing device 500. The touchscreen display 518 includes a touchscreen panel 522 that is internal to, or externally connected to, the computing device 500. For example, the touchscreen panel 522 may be positioned in front of or behind the visual display 520. In some examples, the touchscreen panel 522 is integrated with the visual display screen panel.

The computing device 500 also includes a storage device 524. The storage device 524 is a physical memory such as a hard drive, an optical drive, a thumbdrive, an array of drives, a solid-state drive, or any combinations thereof. The storage device 524 may also include remote storage drives.

The computing device 500 may also include a network interface controller (NIC) 526. The NIC 526 may be configured to connect the computing device 500 through the bus 506 to a network 528. The network 528 may be a wide area network (WAN), local area network (LAN), or the Internet, among others. In some examples, the device may communicate with other devices through a wireless technology. For example, the device may communicate with other devices via a wireless local area network connection. In some examples, the device may connect and communicate with other devices via Bluetooth® or similar technology.

The computing device 500 further includes a thermometer 530. For example, the thermometer 530 may include one or more thermal sensors.

The computing device 500 further includes a camera 532. For example, the camera 532 may include one or more imaging sensors. In some examples, the camera 532 may include a processor to generate images. In various examples, the images may be used to determine the size of a body part. In some examples, the images can be used to detect special cases of body parts that may not fit into typical body part models.

The storage 524 of computing device 500 further includes a print generator 534, a body part measurer 536, a recommendation generator 538, and a size verifier 540. In various examples, each of the print generator 534, the body part measurer 536, the recommendation generator 538, and the size verifier 540 may be a microcontroller, embedded processor, or software module. In some embodiments, the print generator 534 is used to generate digital prints corresponding to body parts presented at the touchscreen panel 522. For example, the print generator 534 can generate digital prints of body parts based on received touch capacitance information. The body part measurer 536 can use digital prints from a specific personal electronic device touchscreen panel to measure various different body parts. In some embodiments, the body part measurer 536 can include one or more body part models, which may be trained neural networks. In various embodiments, the recommendation generator 538 generates recommendations based on the generated digital prints or the measured sizes of the body parts. For example, the recommendations may include shoe sizes, glove sizes, condom sizes, etc. In some embodiments, the sizer verifier 540 verifies received purported sizes of body parts with the measured sizes. For example, a received purported size of a body part from a user may be compared with the determined size of the body part. In various examples, the purported size is verified if the purported size is at least, at most, or within a range of the size determined based on the touch capacitance information.

The block diagram of FIG. 5 is not intended to indicate that the computing device 500 is to include all of the components shown in FIG. 5. Rather, the computing device 500 can include fewer or additional components not illustrated in FIG. 5, such as additional buffers, additional processors, and the like. The computing device 500 may include any number of additional components not shown in FIG. 5, depending on the details of the specific implementation. Furthermore, any of the functionalities of the print generator 534, the body part measurer 536, the recommendation generator 538, or the size verifier 540, may be partially, or entirely, implemented in hardware and/or in the processor 502. For example, the functionality may be implemented with an application specific integrated circuit, in logic implemented in the processor 502, or in any other device. In addition, any of the functionalities of the CPU 502 may be partially, or entirely, implemented in hardware and/or in a processor. For example, the functionality of the print generator 534, the body part measurer 536, the recommendation generator 538, or the size verifier 540 may be implemented with an application specific integrated circuit, in logic implemented in a processor, in logic implemented in a specialized graphics processing unit such as the GPU 508, or in any other device.

FIG. 6 is a block diagram showing computer readable media 600 that store code for measuring body parts using touchscreen displays. As used herein, computer readable media are non-transitory computer readable media. The computer readable media 600 may be accessed by a processor 602 over a computer bus 604. Furthermore, the computer readable medium 600 may include code configured to direct the processor 602 to perform the methods described herein. In some examples, the computer readable media 600 may be storage media.

The various software components discussed herein may be stored on one or more computer readable media 600, as indicated in FIG. 6. For example, a digital print generator module 606 may be configured to generate digital prints based on received touch capacitance information. A body part sizer module 608 may be configured to determine a size of a body part based on the detected touch capacitance information. In some embodiments, the body part sizer module 608 may be configured to measure a generated digital print to determine the size of a corresponding body part. A recommendation module 610 may be configured to generate a recommendation based on the digital print or determined size of a body part. For example, the recommendation may include a recommended shoe size for a foot, or a recommended glove size or a particular hand. A verification module 612 may be configured to receive a purported size of a body part and verify the size of the body part based on the digital print or touch capacitance information. For example, the verification module 612 may be configured to verify the size of a foot for purposes of shoe sizing. In some embodiments, the verification module 612 may be configured to verify that a body part is at least, at most, or within a range of a purported size. For example, a user may use the verification to provide proof of the size of their hand, foot, or other extremities.

The block diagram of FIG. 6 is not intended to indicate that the computer readable media 600 is to include all of the components shown in FIG. 6. Further, the computer readable media 600 may include any number of additional components not shown in FIG. 6, depending on the details of the specific implementation.

### Example Pseudocode for Foot Measurement Application

To generate a digital print of a foot and enable measurement at any point along the y-axis, the following example pseudocode tracks and displays touch points received from a touchscreen panel as the user moves their foot onto a touchscreen display:

### 1. Initialize Application:

### Variables:

‘footOutline’: An array to store touch points for outlining the foot. Multiple touch points are tracked accurately to draw a smooth outline of the foot.
‘yAxisMeasurements’: To store measurements taken at different points along the y-axis.

### Elements:

‘outlineCanvas’: A ‘<canvas>’ element to draw the outline of the foot. The HTML5 ‘<canvas>’ can be used for real-time drawing of the foot outline as the user moves their foot.
‘measurementPoints’: A list or array to store and display measurements along the y-axis.

### 2. Handle Touch Events With Outline:

‘startTouch(event)’
   Capture the starting touch coordinates (‘startX’, ‘startY’).
   Initialize ‘footOutline’ with the first touch point.
‘movetouch(event)’
   Prevent default behavior (e.g., scrolling) during touch movement.
   Update ‘endX’ and ‘endY’ with the current touch coordinates.
   Append the current touch point to ‘footOutline’.
   Redraw the outline of the foot on the ‘outlineCanvas’.
‘endtouch(event)’
   Calculate the width at various points along the y-axis using the stored touch points in ‘footOutline’.
   Store the measurements in ‘yAxisMeasurements’.
   Display the y-axis measurements alongside the foot outline.
   Trigger the ‘showPopup(width, length)’ function to display the final results in a popup.

### 3. Drawing the Foot Outline:

‘drawOutline( )’
   Use the ‘fingerOutline’ array to plot the foot's outline on the ‘outlineCanvas’.
   Continuously update the canvas as the user moves their foot.

### 4. Measuring Along the Y-Axis:

‘measureYAxis( )’
   Iterate through the ‘footOutline’ array.
   Calculate the width at different y-coordinates.
   Store these measurements in ‘yAxisMeasurements’.
   Update the display to show these measurements alongside the y-coordinates.

### 5. Displaying Y-Axis Measurements:

‘displayYAxisMeasurements( )’
   Dynamically create elements or use a list to display the measurements taken at various y-coordinates.
   Align these measurements with the corresponding points on the foot outline.

### 6. Popup and Reset Functionality:

Functionality remains the same as in the initial pseudocode, but the popup may also display the y-axis measurements if required.

### Example Process of Generation of Digital Prints Using Y-axis Measurements:

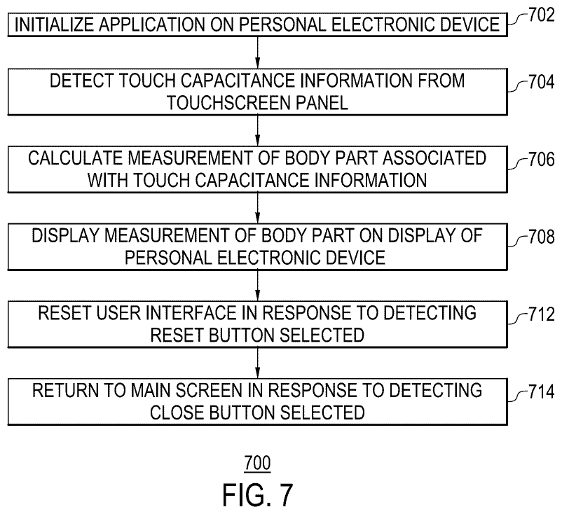

FIG. 7 is a process flow diagram illustrating an example process for generating a digital print of a foot. The example process 700 can be implemented in the system 100 of FIG. 1, the computing device 500 of FIG. 5, or the computer readable media 600 of FIG. 6.

At block 702, an application on a personal electronic device is initialized. For example, initializing the application may include loading the application, initializing variables, and setting up user interface elements. In some examples, setting up the user interface may include setting up a canvas for the foot outline.

At block 704, touch capacitance information is detected from a touchscreen panel. For example, the touchscreen panel may be of the personal electronic device. In some examples, the application may also receive a user selected measurement unit, such as centimeters or inches. In various examples, the user places and moves their foot on a touch area of the touchscreen panel. In some examples, the application draws the foot outline on the canvas and records the touch points.

At block 706, a measurement of the body part associated with the touch capacitance information is calculated. In some examples, measurements are calculated along the y-axis and displayed alongside the foot outline.

At block 708, the measurement of the body part is displayed on the display of the personal electronic device. For example, a final width and length may be displayed, along with a detailed measurement breakdown at various y-axis points. In some examples, a popup box with the measurement summary is also displayed.

At block 710, the user interface is reset in response to detecting that a reset button has been selected. For example, a user may click a reset button to clear the measurements and reset the user interface.

At block 712, the application returns to a main screen in response to detecting that a close button has been selected. For example, a user can close the popup box to return to the main screen.

This process flow diagram is not intended to indicate that the blocks of the example process 700 are to be executed in any particular order, or that all of the blocks are to be included in every case. Further, any number of additional blocks not shown may be included within the example process 700, depending on the details of the specific implementation.

Not all components, features, structures, characteristics, etc. described and illustrated herein need be included in a particular aspect or aspects. If the specification states a component, feature, structure, or characteristic “may”, “might”, “can” or “could” be included, for example, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

It is to be noted that, although some aspects have been described in reference to particular implementations, other implementations are possible according to some aspects. Additionally, the arrangement and/or order of circuit elements or other features illustrated in the drawings and/or described herein need not be arranged in the particular way illustrated and described. Many other arrangements are possible according to some aspects.

In each system shown in a figure, the elements in some cases may each have a same reference number or a different reference number to suggest that the elements represented could be different and/or similar. However, an element may be flexible enough to have different implementations and work with some or all of the systems shown or described herein. The various elements shown in the figures may be the same or different. Which one is referred to as a first element and which is called a second element is arbitrary.

It is to be understood that specifics in the aforementioned examples may be used anywhere in one or more aspects. For instance, all optional features of the computing device described above may also be implemented with respect to either of the methods or the computer-readable medium described herein. Furthermore, although flow diagrams and/or state diagrams may have been used herein to describe aspects, the techniques are not limited to those diagrams or to corresponding descriptions herein. For example, flow need not move through each illustrated box or state or in exactly the same order as illustrated and described herein.

The present techniques are not restricted to the particular details listed herein. Indeed, those skilled in the art having the benefit of this disclosure will appreciate that many other variations from the foregoing description and drawings may be made within the scope of the present techniques. Accordingly, it is the following claims including any amendments thereto that define the scope of the present techniques.

What is claimed is:

1. An apparatus for measuring human body parts, comprising:
a touchscreen display to detect touch capacitance information associated with a body part; and
a processor to determine a size of the body part based on the detected touch capacitance information.

2. The apparatus of claim 1, wherein the processor is to generate a recommendation based on the measured size of the body part.

3. The apparatus of claim 1, wherein the processor is to verify a received purported size of the body part based on a comparison with the measured size of the body part.

4. The apparatus of claim 1, wherein the processor is to:
generate a digital print based on the touch capacitance information; and
measure the generated digital print to determine the size of the body part.

5. The apparatus of claim 4, wherein the digital print comprises a digital footprint or a digital handprint.

6. The apparatus of claim 1, comprising a camera to capture an image of the body part, wherein the size of the body part is determined based on both the detected touch capacitance information and the captured image.

7. The apparatus of claim 1, wherein the body part comprises an extremity.

8. The apparatus of claim 1, comprising disabling a print receival display in response to detecting that the temperature of the touchscreen display exceeds a threshold.

9. The apparatus of claim 1, comprising displaying a print receival display on the touchscreen in response to detecting a body part measurement request.

10. The apparatus of claim 1, comprising a neural network trained using back propagation and gradient descent on training data comprising capacitance information associated with various verified body part measurements, wherein the verified body part measurements are used as ground truth data during training.

11. A method for measuring human body parts, comprising:
detecting, via a touchscreen display of a personal electronic device, at a processor, touch capacitance information associated with a body part; and
determining, via the processor, a size of the body part based on the detected touch capacitance information.

12. The method of claim 11, wherein determining the size of the body part comprises:
generating, via a processor, a digital print of a body part based on the touch capacitance information; and
determining, via the processor, the size of the body part based on the generated digital print.

13. The method of claim 11, wherein determining the size of the body part comprises detecting a discrepancy at one side of the body part relative to an opposing side of the body part.

14. The method of claim 11, wherein determining the size of the body part comprises:
inputting the touch capacitance information into a trained neural network, wherein the neural network is trained using back propagation and gradient descent on training data comprising capacitance information associated with various verified body part measurements; and
receiving the size of the body part from the trained neural network.

15. The method of claim 11, comprising presenting a digital print receiver display on the touchscreen display, and detecting the touch capacitance information within the digital print receiver display.

16. The method of claim 15, comprising disabling the digital print receiver screen in response to detecting that a temperature of the touchscreen display exceeds a threshold.

17. The method of claim 11, comprising receiving a purported size of the body part and verifying the size of the body part based on the digital print.

18. The method of claim 11, wherein the digital print comprises a digital footprint or a digital handprint.

19. The method of claim 11, comprising capturing, via a camera of the personal electronic device, an image of the of the body part, wherein the size of the body part is determined based on both the detected touch capacitance information and the captured image.

20. At least one computer readable medium for measuring human body parts having instructions stored therein that, in response to being executed on a computing device, cause the computing device to:
detect touch capacitance information associated with a body part; and
determine a size of the body part based on the detected touch capacitance information.
